(19) United States
(12) Patent Application Publication
(10) Pub. No.: US 2024/0285233 A1
(43) Pub. Date: Aug. 29, 2024

(54) SYSTEMS AND METHODS FOR MANAGING TISSUE METABOLIC ADEQUACY FOR PERFORMANCE HEALTH AND DISEASE

(71) Applicant: UNIVERSITY OF PITTSBURGH - OF THE COMMONWEALTH SYSTEM OF HIGHER EDUCATION, Pittsburgh, PA (US)

(72) Inventor: Michael R. Pinsky, Pittsburgh, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 18/625,333

(22) Filed: Apr. 3, 2024

Related U.S. Application Data
(63) Continuation of application No. PCT/US2022/049966, filed on Nov. 15, 2022.
(60) Provisional application No. 63/279,538, filed on Nov. 15, 2021.

(51) Int. Cl. A61B 5/00

(52) U.S. Cl. CPC A61B 5/4884; A61B 5/7267; A61B 5/7292

(57) ABSTRACT

A system includes a processor comprising a multivariate predictive model. The multivariate predictive model can be configured to classify responses of a plurality of subjects to a stimulus, predict at least one of a response or an intervention of a target subject to the stimulus based on the classified responses, and adjust a tissue metabolic adequacy of the target subject based on at least one of the predicted response or the predicted intervention of the target subject. The stimulus can include a physical stress, a disease-related stress, a severity of stress, a severity of insult, an external environment condition, or combinations thereof. The tissue metabolic adequacy can be calculated based on a vasomotor tone, a ventricular pump function, an effective circulatory volume, or combinations thereof.

### CROSS-REFERENCE TO RELATED APPLICATIONS

This application is a continuation of International Application No. PCT/US2022/049966, filed on Nov. 15, 2022, which claims priority to U.S. Provisional Application Ser. No. 63/279,538, filed on Nov. 15, 2021, the contents of which are incorporated by reference herein in their entireties.

### STATEMENT REGARDING FEDERALLY-SPONSORED RESEARCH

This invention was made with government support under grant nos. R01-HL-073198 and K24-HL67181 awarded by the National Heart, Lung, and Blood Institute (NHLBI), and grant no. W81XWH19C0101 awarded by U.S. Army Medical Research Acquisition Activity (USAMRAA). The government has certain rights in the invention.

### FIELD

The presently disclosed subject matter relates to techniques for managing the cardiovascular system of a subject.

### BACKGROUND

The human body can have multiple cardiorespiratory compensatory mechanisms that can be utilized to maintain tissue metabolic adequacy. However, there is no specific set of optimal universal setpoints or ranges for these compensatory mechanisms for all humans in all instances.

Rather, these compensatory mechanisms can have patient-specific operating ranges. Furthermore, it is the aggregate effect of these mechanisms that enables the body to achieve a tissue metabolic adequacy state.

Therefore, there remains a need in the art for improved controlling techniques for managing the cardiovascular system of a specific subject for performance health and/or disease.

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

The disclosed subject matter provides systems and methods for monitoring and/or managing the cardiovascular system of a subject. An example system can include a processor including a multivariate predictive model. The multivariate predictive model can be configured to classify responses of a plurality of subjects to a stimulus, predict at least one of a response or an intervention of a target subject to the stimulus based on the classified responses, and adjust a tissue metabolic adequacy of the target subject based on at least one of the predicted response or the predicted intervention of the target subject. In certain embodiments, the stimulus can include a physical stress, a disease-related stress, a severity of stress, a severity of insult, an external environment condition, or combinations thereof. In non-limiting embodiments, the tissue metabolic adequacy can be calculated based on a vasomotor tone, a ventricular pump function, an effective circulatory volume, or combinations thereof.

In certain embodiments, the external environment condition can include altitude, temperature, humidity, gravity, or combinations thereof. In non-limiting embodiments, at least one of the response or the intervention of the target subject can include a phenotype ranging from a robustness to a fragility factor of the subject.

In certain embodiments, the processor can be configured to calculate a tissue metabolic adequacy operating region based on at least one of the responses, the stimulus, the vasomotor tone, the ventricular pump function, the effective circulatory volume, or combinations thereof.

In certain embodiments, the system can further include a medical device that monitors and/or maintains the cardiovascular stability of the subject.

In certain embodiments, the system can include an open-loop feedback control system. In non-limiting embodiments, the open-loop feedback control system can be configured to measure and adjust at least one of the vasomotor tone, the ventricular pump function, the effective circulatory volume, or combinations thereof.

In certain embodiments, the system can include at least one sensor that can sense the external environment condition.

In certain embodiments, the multivariate predictive model can be configured to be trained by a machine learning algorithm.

The disclosed subject matter provides computer-implemented methods for monitoring and/or managing the cardiovascular system of a subject. An example method can include classifying, using a multivariate predictive model, responses of a plurality of subjects to a stimulus; predicting at least one of a response or an intervention of a target subject to the stimulus based on the classified responses; and adjusting a tissue metabolic adequacy of the target subject based on at least one of the predicted response or the predicted intervention of the target subject. In non-limiting embodiments, the stimulus can include a physical stress, a disease-related stress, a severity of stress, a severity of insult, an external environment condition, or combinations thereof.

In certain embodiments, the stimulus can comprise the external environment condition. The external environment condition can include at least one of altitude, temperature, humidity, gravity, or combinations thereof. In non-limiting embodiments, the method can include sensing the external environment condition using at least one sensor.

In certain embodiments, the response or the intervention of the target subject can include a phenotype ranging from a robustness to a fragility of the subject.

In certain embodiments, the disclosed method can include calculating the tissue metabolic adequacy operating region based on the responses, the stimulus, the vasomotor tone, the ventricular pump function, the effective circulatory volume, or combinations thereof.

In certain embodiments, the disclosed method can include maintaining a cardiovascular stability of the subject using an open-loop feedback control system. In non-limiting embodiments, the open-loop feedback control system can be configured to measure and adjust at least one of a vasomotor tone, a ventricular pump function, an effective cardiac ejection, an effective circulatory volume, or combinations thereof.

In certain embodiments, the disclosed method can include training the multivariate predictive model using a machine learning algorithm.

In certain embodiments, the tissue metabolic adequacy of the target subject is adjusted by administering an effective amount of an active agent.

### BRIEF DESCRIPTION OF THE DRAWINGS

FIG. 1 is a three-dimensional graph illustrating that the tissue metabolic adequacy operating region as a function of vasomotor tone (VT), effective cardiac ejection (EE), and effective circulatory volume (ECV) and showing that the disclosed device can be agnostic as to the bio-signals from each of these specific parameters that can be derived in accordance with the disclosed subject matter.

FIG. 2 is a three-dimensional graph showing a phenotype classification of physiological robustness or fragility in accordance with the disclosed subject matter.

FIGS. 3A-3F depict graphs showing metabolic adequate operating ranges that can be affected by the form and degree of stress in accordance with the disclosed subject matter.

FIG. 4 is a diagram showing the ability of the disclosed system to store and manage behaviors in a database, catalog, or registry where pre-defined behaviors can be used as a library of predictive behaviors to enable recognition by the medical device to aid in managing a subject in accordance with the disclosed subject matter.

FIG. 5 is a flow diagram of the system in accordance with the disclosed subject matter.

### DETAILED DESCRIPTION

As used herein, the following terms have the meanings ascribed to them below unless specified otherwise. Abbreviations used herein have their conventional meaning within the chemical and biological arts.

As used in the specification and the appended claims, the singular forms “a,” “an,” and “the” include plural referents unless the context clearly dictates otherwise. Thus, for example, a reference to “a compound” includes mixtures of compounds.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within three or more than three standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “active agent” refers to an agent that is capable of having a physiological effect when administered to a subject. In certain embodiments, the term “active agent” refers to an agent that can improve or maintain the cardiovascular system of a subject. For example, the active agent can include any drugs or agents that can control vasomotor tone, effective cardiac ejection, or effective circulatory volume.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of,” and “consisting essentially of,” the embodiments or elements presented herein, whether explicitly set forth or not.

The term “effective amount,” as used herein, refers to the amount of active agent sufficient to treat, prevent, or manage a disease. Further, a therapeutically effective amount with respect to the second targeting probe of the disclosure can mean the amount of active agent alone or in combination with other therapies that provide a therapeutic benefit in the treatment or management of the disease, which can include a decrease in the severity of disease symptoms, an increase in frequency and duration of disease symptom-free periods, or a prevention of impairment or disability due to the disease affliction. The term can encompass an amount that improves overall therapy, reduces or avoids unwanted effects, or enhances the therapeutic efficacy of or synergies with another therapeutic agent.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either endpoint of the range are specifically contemplated. For example, a nested sub-range of an exemplary range of 1 to 50 can include 1 to 10, 1 to 20, 1 to 30, and 1 to 40 in one direction, or 50 to 40, 50 to 30, 50 to 20, and 50 to 10 in the other direction.

A “subject” may be a human or a non-human animal, for example, but not by limitation, a non-human primate, a dog, a cat, a horse, a rodent, a cow, a goat, a rabbit, a mouse, etc.

The terms “treat,” “treating,” or “treatment,” and other grammatical equivalents as used herein include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit and/or a prophylactic benefit. Therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder.

The disclosed subject matter provides techniques for managing the cardiovascular system of a specific human or animal for performance health and/or disease. The disclosed techniques can be employed by a medical device to aid in managing the cardiovascular system of a subject for performance health and/or disease.

In certain embodiments, an example system can include a processor, which can be configured to employ a multivariate model predictive control approach to manage cardiovascular stability. The disclosed system can be configured to apply a process control paradigm toward managing cardiovascular health where the cardiovascular system operates in a multi-dimensional (or multivariate) operating region bounded by tissue metabolic adequacy. The tissue metabolic adequacy can be defined as a state of target tissues that can maintain the healthy or normal cardiovascular system.

In certain embodiments, the disclosed processor can be configured to assess or consider dependent process variables and independent process variables. In certain embodiments, the dependent process variables can include the cardiovascular compensatory mechanisms (e.g., vasomotor tone, ejection effectiveness, and effective system circulatory blood volume) that enable the body to adapt the cardiovascular function to stress to maintain cellular metabolic adequacy. The independent variables, which cannot be adjusted, can include the human or animal physiological phenotype that can determine the robustness or fragility (e.g., frailty) of a specific subject's cardiovascular system to adapt to physical or disease-related stress.

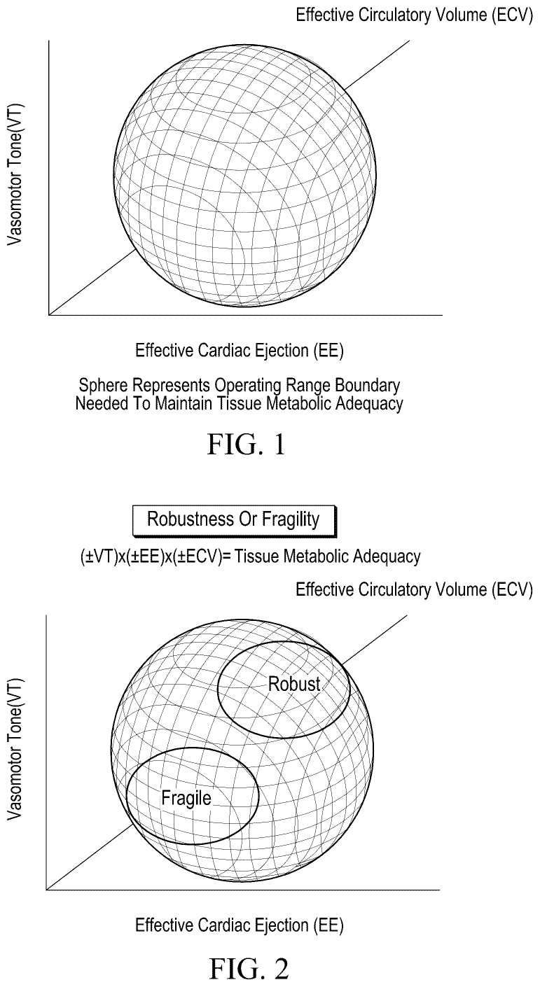

In certain embodiments, the disclosed system can quantify the tissue metabolic adequacy operating region and manage as the product of the contributions from the cardiovascular compensatory mechanisms. For example, as shown in FIG. 1, the compensatory mechanisms, which enable the body to achieve a tissue metabolic adequacy state, can be defined by vasomotor tone (VT), ventricular pump function (also known as ejection effectiveness (EE)), and the effective system circulatory blood volume (ECV). The control of the cardiovascular system is closely linked to metabolic demand, as the cardiovascular system serves to allow for normal metabolic function and response to transient stresses as an acute adaptive process. Chronic stresses can result in the remodeling of the heart, vessels and set points of their controllers for blood pressure, contractility and blood volume.

In certain embodiments, the VT, EE, and ECV can be measured separately, but their interactions provide much more insight and allow for more acute identification of disease onset and end points for resuscitation once sufficiency has been achieved. For example, although mean circulatory filling pressure and its changes can be measured as a measure of effective circulating blood volume, left ventricular power (e.g., stroke volume×ejection pressure) as a measure of left ventricular systolic function and dynamic arterial elastance (the ratio of pulse pressure variation to stroke volume variation over 15-20 seconds), it is their dynamic interactions that define health, robustness and their antithesis disease and fragility (e.g., frailty).

The sphere in FIG. 1 represents the operating range boundaries of VT, ECV, and EE for maintaining tissue metabolic adequacy. In non-limiting embodiments, the operating range boundaries of VT, ECV, and EE can be adjusted based on each subject's conditions (e.g., the health of the cardiovascular system) or normal ranges of a subject's cardiovascular system known in the art. A cardiovascular stability can be defined as an operating region (e.g., the inside of the three-dimensional sphere in FIG. 1) where the body's tissue metabolic adequacy has been met. For example, cardiovascular stability can be achieved where the states of cardiovascular parameters are consistent with adequate blood flow to meet the metabolic demands of the body without being in overt failure. In non-limiting embodiments, the cardiovascular parameters can include an oxygen delivery rate (e.g., a mixed venous oxygen saturation level), metabolic acidosis rate (e.g., serum lactate level), a heart rate, or a combination thereof.

In certain embodiments, the disclosed system can be agnostic as to the bio-signals from each of these specific parameters can be derived. For example, the disclosed system can include a medical device that can be agnostic in regard to sensor modality, as these cardiovascular compensatory measures can be derived from a host of invasive or non-invasive sensor modalities. The tissue metabolic adequacy operating range as the product of the compensatory contributions (e.g., VT, EE, and ECV) can be quantified using the following equation (1) and can be represented as a three-dimensional graph, as shown in FIG. 1.

$$( \pm V T ) \times ( \pm EE ) \times ( \pm ECV ) = Tissue Metabolic Adequacy \qquad ( 1 )$$

In non-limiting embodiments, the disclosed system and graph can be used for the disclosed system to be agnostic as to the bio-signals from each of the specific parameters.

In certain embodiments, the disclosed system can assess various factors related to tissue metabolic adequacy. For example, the disclosed system can consider various factors that can determine the degree to which each of these compensatory contributions can be applied and their appropriate operating range. In non-limiting embodiments, the factor related to the tissue metabolic adequacy can include a patient phenotype, a form of stress or insult, a severity of stress or insult, a context, an environment, or combinations thereof. The phenotype can range from a patient's physiological robustness or fragility. In non-limiting embodiments, the patient's physiological robustness factor or fragility factor can be determined based on age, genetics, pre-existing disease state, or combinations thereof. Robustness can be measured by a high degree of the intrinsic variability of baseline signals around a mean that can also adapt to external challenges like exercise, infection, and bleeding. Fragility is the absence of robustness. The context or the environment can include altitude, temperature, humidity, transport type effects (e.g., hyper-gravity), or combinations thereof.

In certain embodiments, the disclosed processor can be configured to consider dependent process variables and independent process variables. The independent variables, which cannot be adjusted, can include the human or animal physiological phenotype that can determine the robustness or fragility factors of a specific subject's cardiovascular system to adapt to physical or disease-related stress.

In certain embodiments, the disclosed processor, using the multivariate predictive model, can be configured to classify the responses of a plurality of subjects to a stimulus. In non-limiting embodiments, the stimulus can be a physical stress, a disease-related stress, a form of stress, a severity of stress, a form of insult, a severity of insult, an external environment condition, or combinations thereof. In non-limiting embodiments, the external environment condition can include altitude, temperature, humidity, gravity, other types of environmental conditions that the subject is exposed to, or combinations thereof.

As an example, FIG. 2 shows a phenotype classification of physiological robustness or fragility. The phenotype can be based on a subject's resilience or reserve to adapt to stress. A low fragile factor status can be found when one or more of the compensatory mechanisms is compromised. For example, a patient with a low fragile factor can barely maintain stability and has a longer duration to adapt or respond to an intervention. The phenotype classification of physiological robustness or fragility can include factors such as age, genetics, and pre-existing or chronic diseases. These phenotype classifications can become evident when assessing how different acute patients deteriorate in an ICU setting based on monitoring current physiology parameters and then applying data mining techniques to identify commonalities within the subject's medical records.

For example, the phenotype can be based on the subject's response to stress or an intervention. These factors can determine the fragility or robustness factor of the subject's response, which in part includes how quickly the subject responds. Alternatively, phenotypes can be predicted based on specific attributes within the subject's medical record that include age, genetics, and pre-existing disease states that have been correlated to specific stress response behaviors. One of the benefits of employing a stress response behavior to determine a subject's phenotype can be the ability to manage the subject using compensatory measures derived from non-invasive physiological sensors without the need to capture a subject's baseline.

In non-limiting embodiments, the disclosed system can provide improved accuracy. For example, the disclosed system can include non-invasive sensors that can provide relative changes, and the disclosed process can assess the degree of compensation using the non-invasive sensors by comparing the current measure against a measure previously captured from the subject under healthy non-stressed conditions.

In certain embodiments, the independent variables can include the form and severity of acute stress or insult or a combination thereof. Acute stress can be the result of changes within the body resultant from disease progression, physical activity and related exertion, or an external insult that can result in an injury that compromises cardiovascular function.

In certain embodiments, the tissue metabolic adequate operating range can also be affected by the form and degree of stress. When a specific subject with a phenotype classification encounters a specific acute or current form of physiological stress from disease, insult, or physical activity, the ability of that individual to adapt can be portrayed by their operating range. Fragile phenotypes can demonstrate a limited capability to adapt, while robust phenotypes can have ample resilience or capacity to adapt. The capacity of these phenotypes to adapt is evident in how individuals deteriorate from an insult or disease, as shown by the different groupings of patients in an ICU setting. It is also evident in the progression of how patients respond to treatment.

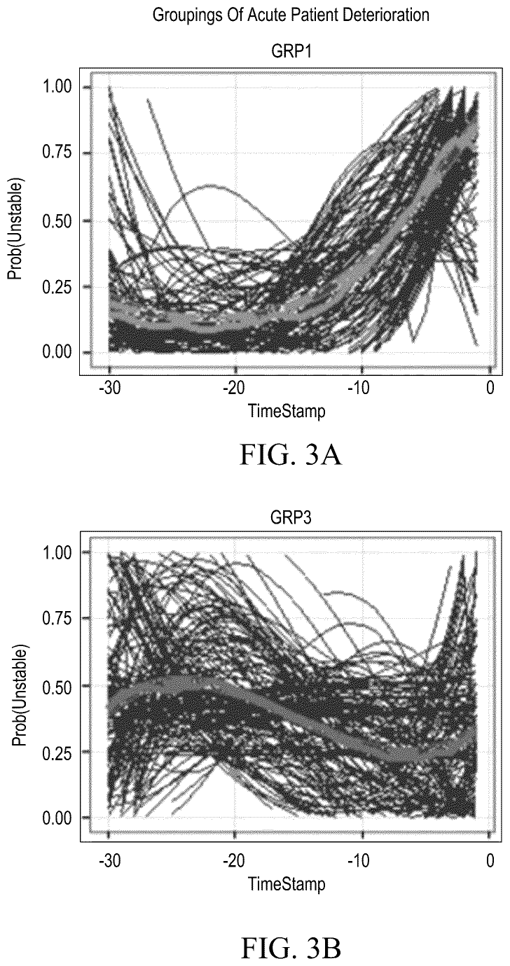

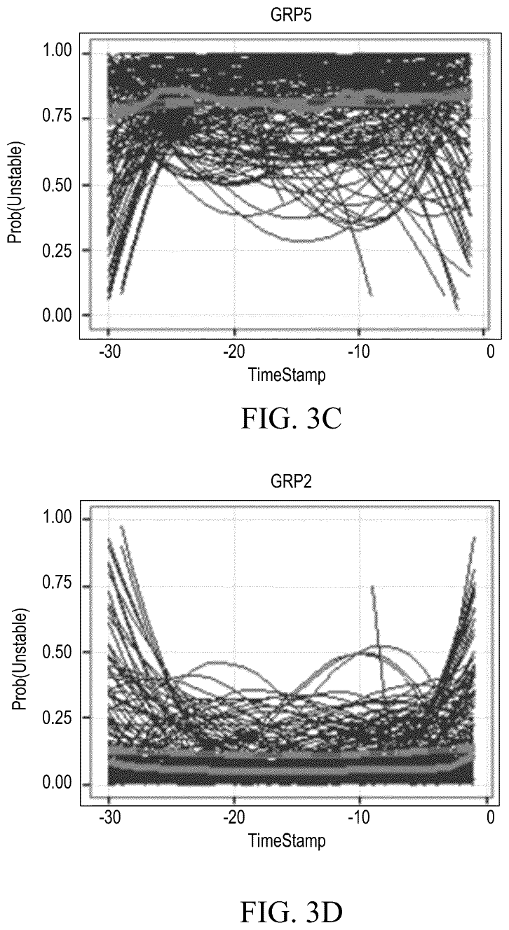

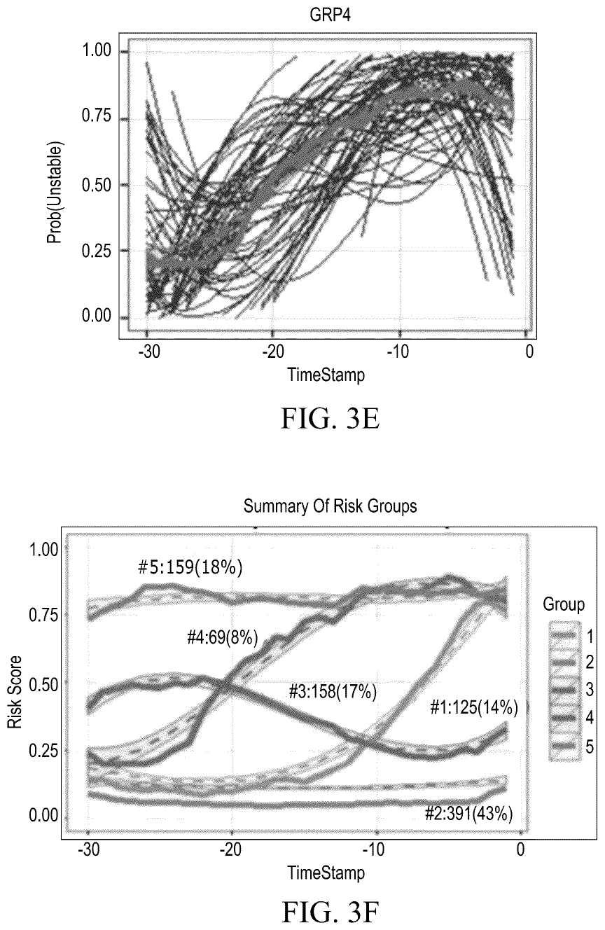

FIGS. 3A-3F depict graphs showing metabolic adequate operating ranges that can be affected by the form and degree of stress in accordance with the disclosed subject matter. For example, FIGS. 3A-3F provide examples of trajectories of progression to insufficiency in a large cohort of patients who progress from stable to instability over 30 minutes. FIG. 3F presents 5 groups of risk process trajectories learned from a dataset. Each summarizes cardio-respiratory instability (CRI) risk progression for the cross-validated set of patients during 30 minutes before the onset of their CRI event (or nonevent for the controls). Solid lines in FIG. 3F show the representative risk process trajectories for each group aggregated over the individual risk trajectories underlying the particular group, where the group assignment is based on their maximum posterior probability of group membership given the full length of trajectories. Dotted lines depict means and confidence intervals estimated from the trajectory group models. On the horizontal axis, timestamp 0 indicates the onset of CRI. The vertical axis scale shows the risk scores. The graph also includes group size information (with count on the left and proportion in parenthesis) obtained from the GBM model.

FIGS. 3A-3E show the most likely trajectories for each of the group-based risk process models, overlaid with the corresponding individual risk processes, each smoothed with third-order polynomials. When FIGS. 3A-3E are combined, the combined graph can reflect the entire cohort, and it shows that individual patient trajectories are independent of others. However, by latent subgroup analysis using the disclosed machine learning tool, there are certain different trajectories. For example, Group 1 in FIG. 3A appears normal but starts to deteriorate approximately 10 minutes before decompensation, Group 3 in FIG. 3B becomes unstable, attempts to compensate and fails, Group 5 in FIG. 3C is always unstable and merely stops trying, Group 2 in FIG. 3D is the stable group that never decompensated, and Group 4 in FIG. 3E decompensated at ˜20 minutes. Importantly these subgroups are not always disease-specific but patient-specific, meaning that if they decompensate a second time, it is on this same pathway even though the biological causes of decompensation can be respiratory, cardiac reserve or sepsis.

FIGS. 3A-3F also graphically demonstrate the measures of sufficiency and insufficiency that the disclosed monitoring modality is agnostic, allowing the disclosed subject matter to be applied to any monitoring techniques. In non-limiting embodiments, the risk score in FIGS. 3A-3F can be calculated by the disclosed classification techniques. For example, the trained random forest model can be used to score the data observed during periods preceding the onset of CRI or control non-events, separately and independently for each of the 30 reference timestamps, and for each of the patient/event combination from a held out data set that was not used to train the classifier to mitigate the risk of over-fitting.

In certain embodiments, the predictions made by the trained classifier on the held-out data served as the estimated risk scores. Their values can range from approximately 0.0 to approximately 1.0, with the higher values reflective of a greater similarity of the currently observed vital signs features to those of patients going into an episode of CRI, and conversely, lower values suggest resemblance to the non-events obtained from control patients. Computing these scores over time at one-minute intervals, time series of scores that can be used to reflect the risk process as it is leading towards the event of interest can be obtained. The temporal sequences of the estimated risk scores can be used to learn a finite mixture model of risk score trajectories (e.g., third-order polynomials).

In certain embodiments, the independent variable can include the context or external environment in which a subject (e.g., humans or animals) can be subjected. These variables can include altitude, temperature, humidity, and other types of effects, such as hyper-gravity experienced during patient or subject transport. In non-limiting embodiments, the form and severity of stress, as well as external factors, can affect the subject's response. The stress response behaviors or signatures can be further segregated by the environmental factors by which they are affected. For example, when a specific phenotype is subjected to acute stress, such as a gunshot that leads to internal bleeding or hemorrhage, the response behavior can also be affected by when the subject is in a tropical environment with high ambient temperatures or in the artic with extremely cold temperatures.

Physiological stress can be resultant of a physical workload that incurs an increased tissue metabolic demand, an insult from disease or injury that compromises a compensatory mechanism, or a form of treatment (e.g., treatment for robust or fragile conditions). Similarly, the subject can be transported in an aggressive manner in a military setting where a rapid rotary-wing transport causes changes in lung perfusion causing loss of lung capacity and further compromising the subject's response behavior. These external factors can affect the behavior and the operating region within the tissue metabolic adequacy operating range.

In certain embodiments, the disclosed system can predict a response or an intervention of a subject to a stimulus based on the classified responses. For example, the disclosed system can include a predictive model that can predict a response or an intervention of a subject to a stimulus based on the classified responses. In non-limiting embodiments, the disclosed predictive models can be based on grouping common patient or subject responses to physiological stress or intervention into classes of response referred to as a patient phenotype. As opposed to employing population health methods where patient record data is mined and correlated to patient outcomes from specific treatment protocols, patient records can be mined to correlate common adaptive behaviors referred to as subject phenotypes in accordance with the embodiments of the disclosed subject matter. These phenotypes can then be used to predict how a subject exposed to acute or physical stress or an intervention-related stress will respond to aid in managing the subject's tissue metabolic adequacy.

In certain embodiments, the disclosed system can be self-learning in regards to a specific subject, in that the system is able to recognize the fragility, robustness, the subject's changes, or combinations thereof over time in response to disease and treatment, thus making the type and magnitude of care personalized and precise. In non-limiting embodiments, the multivariate predictive model can be trained by a machine learning technique, and the trained predictive model can be used for predicting the response or the intervention of a target subject to the stimulus and adjusting the tissue metabolic adequacy of the target subject based on the predicted response or the predicted intervention of the target subject. In non-limiting embodiments, any type of machine learning algorithm can provide a numeric score output. For example, the multivariate predictive model can utilize a supervised learning algorithm, an unsupervised learning algorithm, a reinforcement learning algorithm, a semi-supervised learning algorithm, or combinations thereof. In non-limiting embodiments, the machine learning algorithm can be the Random Forest model. In one example, the multivariate predictive model with a machine learning algorithm (e.g., Random Forest model) can be trained using a library of data (e.g., Python sklearn library) to predict the response or the intervention of a target subject (e.g., robustness or fragility) to various stimulus (e.g., stress, insult, environmental factors). In one example, the disclosed predictive model can be trained and evaluated using a leave-one-subject-out cross-validation protocol, in which during each training iteration, one subject can be held-out as the test set, while the data of all the other subjects can be used for training and validation. In non-limiting embodiments, the training data can include a physical stress, a disease-related stress, a form of stress, a severity of stress, a form of insult, a severity of insult, an external environment condition (e.g., altitude, temperature, humidity, gravity, other types of environmental conditions), a robustness of a subject, a fragility of a subject, a vasomotor tone, a ventricular pump function, an effective cardiac ejection, an effective circulatory volume, or combinations thereof.

In non-limiting embodiments, the data for training the multivariate model can be normalized. For example, the data related to the dependent variables and independent variables can be normalized by normalization factors (e.g., the subject's personal baseline established from the subject's stabilization period data: median or 90% ranges of dependent or independent variables). The normalized data can be used for training the multivariate model. In non-limiting embodiments, the disclosed machine learning algorithm can create and utilize a hypotension prediction index (HPI) based solely on the radial arteria pressure waveform featurization over time.

In non-limiting embodiments, the reference to a personal baseline can be collected during the subjects' stable state and can be used for improving the performance of the multivariate predictive model and decreasing the detrimental effects of heterogeneity among the subjects.

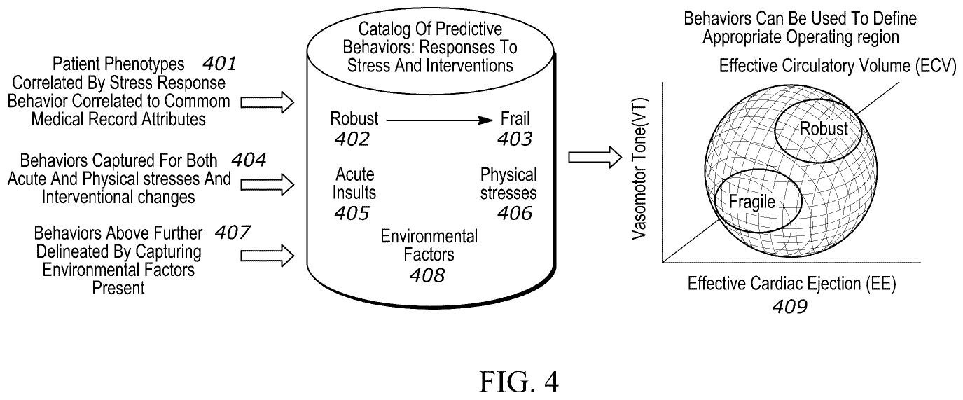

FIG. 4 shows the disclosed system's ability to store and manage behaviors in a database, catalog, or registry where pre-defined behaviors can be used as a library of predictive behaviors to enable recognition by the medical device to aid in managing the subject. For example, the disclosed patient phenotypes correlated by stress response behavior or common medical 401 recorded attributes can be classified (e.g., robustness 402 or fragileness 403) and stored in the disclosed system. Behaviors 404 captured for acute stresses, physical stresses, or intervention changes can be classified (e.g., acute insults 405 or physical stresses 406) and stored in the disclosed system. Behaviors 407 delineated by capturing environmental factors can be stored as environmental factors 408 (e.g., altitude, temperature, humidity, gravity, and other types of environmental conditions) in the system. The stored factors can be further used for predicting a response or an intervention of a target subject to a stimulus. For example, the disclosed system can estimate and define the appropriate operating region for medical devices for the disclosed tissue metabolic adequacy and predict a response or an intervention of a target subject to the stimulus based on the calculated tissue metabolic adequacy and classified responses/factors 409.

In certain embodiments, the disclosed system can include a device that can adjust a tissue metabolic adequacy of the target subject based on the predicted response or the predicted intervention of the target subject. For example, the disclosed system can include a device that can modify the vasomotor tone (VT), ventricular pump function (also known as ejection effectiveness (EE)), and the effective system circulatory blood volume (ECV).

In non-limiting embodiments, the disclosed system can include a medical device that can monitor and/or maintain the cardiovascular stability of the subject. For example, cardiovascular stability can be defined as an operating region (e.g., the insider of the three-dimensional sphere in FIG. 1) where the body's tissue metabolic adequacy has been met. The medical device can monitor whether the status of the subject's metabolic adequacy is inside of the operating region and administer an active agent for adjusting CT, EE, ECV, or a combination thereof if the status of metabolic adequacy is outside of the operating region.

In certain embodiments, the disclosed system can be an open-loop feedback control system. An open-loop system refers to a scenario where a clinician is involved in overseeing the control process and adjusting the dependent variables to maintain tissue metabolic adequacy. A feedback control system can be the system that can compare and adjust the function of the dependent variables to maintain tissue metabolic adequacy. For example, the open-loop feedback control system can be configured to measure and adjust a vasomotor tone, a ventricular pump function, an effective cardiac ejection, an effective circulatory volume, or combinations thereof.

In certain embodiments, the disclosed system can include at least one sensor that can capture the external environment condition. In non-limiting embodiments, the external conditions can such as altitude, humidity, temperature, accelerometry data, a hyper-gravity, or combinations thereof. For example, the disclosed system can include an accelerometer, a gravimeter, a thermometer, a hygrometer, an altimeter, or combinations thereof.

In certain embodiments, with respect to tissue metabolic adequacy, there can be a threshold where adequacy has been met and an operating range for a specific subject. If the subject is within an operating range that barely meets their metabolic adequacy, it can be referred to as acutely stable. If the operating range exceeds the minimum metabolic needs, the subject's cardiovascular system can be considered to be healthy and possess additional reserve or capacity to adapt to stress.

In certain embodiments, the disclosed system can include an acute or a severe chronic care system. The disclosed system can be configured to perform the open-loop control that can be combined with decision support software to aid in determining an optimal interventional protocol.

In certain embodiments, the disclosed system can be configured to perform an acute automated feedback-based control. This application can use feedback to continually adjust the different compensatory mechanisms to the extent each of them can be adjusted.

In certain embodiments, the disclosed system can be configured to an unmanned or manned aerial vehicle transport system. The disclosed system can include sensors that can capture external conditions such as altitude, humidity, and temperature and transport-related measures such as accelerometry data to capture the effects of hyper-gravity.

The presently disclosed subject matter also relates to methods for managing the cardiovascular system of a subject. In non-limiting embodiments, the method can be a computer-implemented method.

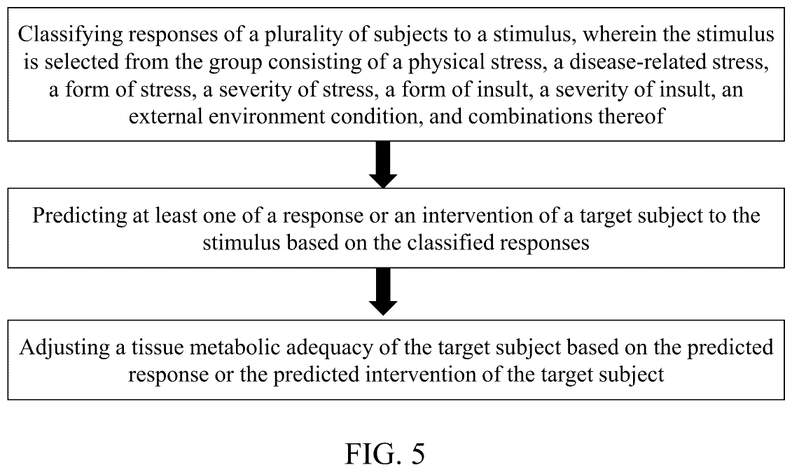

In certain embodiments, as shown in FIG. 5, the computer-implemented method can include classifying, using a multivariate predictive model, responses of a plurality of subjects to a stimulus. In non-limiting embodiments, the stimulus can include a physical stress, a disease-related stress, a form of stress, a severity of stress, a form of insult, a severity of insult, an external environment condition, or combinations thereof. In non-limiting embodiments, the data, including dependent process variables and independent process variables, can be assessed and classified. In one example, the dependent process variables can include the cardiovascular compensatory mechanisms (e.g., vasomotor tone, ejection effectiveness, and effective system circulatory blood volume) that enable the body to adapt the cardiovascular function to stress to maintain cellular metabolic adequacy. The independent variables, which cannot be adjusted, can include the human or animal physiological phenotype that can determine the robustness or fragility of a specific subject's cardiovascular system to adapt to physical or disease-related stress. In non-limiting embodiments, the external environment condition can include altitude, temperature, humidity, gravity, other types of environmental conditions that the subject is exposed to, or combinations thereof. In certain embodiments, the computer-implemented method can further include capturing the external environment condition using at least one sensor.

In certain embodiments, the computer-implemented method can include predicting at least one of a response or an intervention of a target subject to the stimulus based on the classified responses. For example, the phenotype can be based on the subject's response to stress or an intervention. These factors can determine the fragility or robustness of the subject's response, which in part includes how quickly the subject responds. Alternatively, phenotypes can be predicted based on specific attributes within the subject's medical record that include age, genetics, and pre-existing disease states that have been correlated to specific stress response behaviors. In non-limiting embodiments, the response or the intervention of the target subject can include a robustness of the subject, a fragility of the subject, or a combination thereof.

In certain embodiments, the computer-implemented method can include adjusting a tissue metabolic adequacy of the target subject based on at least one of the predicted response or the predicted intervention of the target subject. In non-limiting embodiments, the tissue metabolic adequacy can be calculated based on a vasomotor tone, a ventricular pump function, an effective cardiac ejection, an effective circulatory volume, or combinations thereof. In certain embodiments, the computer-implemented method can include calculating a tissue metabolic adequacy operating region based on the responses, the stimulus, a vasomotor tone, a ventricular pump function, an effective cardiac ejection, an effective circulatory volume, or combinations thereof. In one example, as shown in FIG. 1, the appropriate operating region for maintaining the disclosed tissue metabolic adequacy (e.g., three-dimensional sphere) can be calculated based on the disclosed classified responses/factors.

In certain embodiments, the computer-implemented method can include maintaining a cardiovascular stability of the subject using an open-loop feedback control system. In non-limiting embodiments, the open-loop feedback control system can be configured to measure and adjust a vasomotor tone, a ventricular pump function, an effective cardiac ejection, an effective circulatory volume, or combinations thereof. In non-limiting embodiments, the tissue metabolic adequacy of the target subject is adjusted by administering an active agent.

In certain embodiments, the computer-implemented method can include training the multivariate predictive model using a machine learning algorithm. In non-limiting embodiments, any type of machine learning algorithm with a numeric score output. For example, the multivariate predictive model can utilize a supervised learning algorithm, an unsupervised learning algorithm, a reinforcement learning algorithm, a semi-supervised learning algorithm, or combinations thereof. In non-limiting embodiments, the machine learning algorithm can be the Random Forest model. In one example, the multivariate predictive model with a machine learning algorithm (e.g., Random Forest model) can be trained using a library of data (e.g., Python sklearn library). In one example, the disclosed predictive model can be trained and evaluated using a leave-one-subject-out cross-validation protocol, in which during each training iteration, one subject can be held-out as the test set while the data of all the other subjects can be used for training and validation. In non-limiting embodiments, the training data can include a physical stress, a disease-related stress, a form of stress, a severity of stress, a form of insult, a severity of insult, an external environment condition (e.g., altitude, temperature, humidity, gravity, other types of environmental conditions), a robustness of a subject, a fragility of a subject, a vasomotor tone, a ventricular pump function, an effective cardiac ejection, an effective circulatory volume, or combinations thereof.

In certain embodiments, the computer-implemented method can further include normalizing the training data. The data for training the multivariate model can be normalized. For example, the data related to the dependent variables and independent variables can be normalized by normalization factors (e.g., the subject's personal baseline established from the subject's stabilization period data: median or 90% ranges of dependent or independent variables). The normalized data can be used for training the multivariate model. In non-limiting embodiments, the reference to a personal baseline can be collected during the subjects' stable state and can be used for improving the performance of the multivariate predictive model and decreasing the detrimental effects of heterogeneity among the subjects.

In certain embodiments, the disclosed machine learning techniques can be used to define the specific behaviors or signatures of subjects and their changes over time as patients progress from health to disease and back. Once specific behavior or signatures are defined, they can be transferable across patient groups as all patients have the same overall cardiovascular process and homeostasis control systems. For example, the disclosed machine learning techniques can be used to feature time series data to inform the clinician of exactly the cardiovascular state of the patient and their most likely clinical trajectory. The disclosed machine learning techniques can also be used for predicting future hemodynamic events (e.g., EE, VT, or ECV), such as impending hypotension goes beyond the current practice of monitoring the current state of the patient. Untoward event prediction models are built on a training set using featurization of the specific hemodynamic monitoring data, either every few minutes, beat-to-beat or waveform and then tested on a separate validation set.

The disclosed subject matter can predict the impending hypotension that can occur approximately from 5 minutes to approximately 15 minutes, making them best suited for emergency and intra-operative care environments. The disclosed subject matter can include a Super Learner or supervised machine-learning algorithm or Hybrid Deep Learning models for the detection of hypotension.

In certain embodiments, the disclosed subject matter can be modified or adjusted for predicting different measures. For example, the disclosed subject matter can be used for maintaining cellular metabolic adequacy and/or controlling cardiovascular compensatory mechanisms. In non-limiting embodiments, the disclosed subject matter can provide a multi-dimension process control diagram as the graphical user interface (GUI), which can provide a more accurate real-time understanding of the interdependent relationship of these compensatory mechanisms that together, contributes to this endpoint.

In certain embodiments, a lesser trained caregiver can easily comprehend and act on these deficient compensatory mechanisms based upon the multi-dimension graphic interface as opposed to a complex decision tree. The disclosed system can apply multiple rule-based event monitoring algorithms in a near parallel time period to enable portraying interdependent changes on the desired cellular metabolic endpoint, while the use of decision trees only provides a more gradual sequential understanding of what compensatory mechanisms are deficient.

In certain embodiments, the ability of the disclosed system to query the disclosed algorithms in near parallel time and to handle human process control (e.g., maintaining cellular metabolic adequacy and/or controlling cardiovascular compensatory mechanisms) also allows for a more optimal means of automating control of interventions as these techniques can also account for multiple interdependencies in a more rapid fashion.

In certain embodiments, the disclosed performance health can be defined in the context with physical activities. In certain embodiments, the physical activities can include any type of extreme physical activities where it is important to manage cardiovascular function (e.g., in extreme environmental conditions-high temperatures, mountainous environments, long-duration physical activities where the subject's ability to function is dependent on pro-actively managing their cardiovascular system function). This can also include activities of fighter pilots that can be affected by hyper-gravity and/or high altitude and divers that diver in extreme temperatures and in at significant depths.

All patents, patent applications, publications, product descriptions, and protocols, cited in this specification are hereby incorporated by reference in their entirety. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain, using no more than routine experimentation, many equivalents to the specific embodiments described herein. Such equivalents are intended to be encompassed by the following claims.

Various patents and patent applications are cited herein, the contents of which are hereby incorporated by reference herein in their entirety.

What is claimed is:

1. A system, comprising:
a processor including a multivariate predictive model, the processor configured, using the multivariate predictive model, to
classify responses of a plurality of subjects to a stimulus, wherein the stimulus is selected from the group consisting of a physical stress, a disease-related stress, a severity of stress, an external environment condition, and combinations thereof;
predict at least one of a response or an intervention of a target subject to the stimulus based on the classified responses; and
adjust a tissue metabolic adequacy of the target subject based on at least one of the predicted response or the predicted intervention of the target subject, wherein the tissue metabolic adequacy is calculated based on a vasomotor tone, a ventricular pump function, an effective circulatory volume, or combinations thereof.

2. The system of claim 1, wherein the external environment condition includes altitude, temperature, humidity, gravity or combinations thereof.

3. The system of claim 1, wherein at least one of the response or the intervention of the target subject comprises a phenotype ranging from a robustness to a fragility of the subject.

4. The system of claim 1, wherein the processor is configured to calculate a tissue metabolic adequacy operating region based on at least one of the responses, the stimulus, the vasomotor tone, the ventricular pump function, the effective circulatory volume, or combinations thereof.

5. The system of claim 1, wherein the system further comprises a medical device that monitors and/or maintains cardiovascular stability of the subject.

6. The system of claim 1, wherein the system comprises an open-loop feedback control system, wherein the open-loop feedback control system is configured to measure and adjust at least one of the vasomotor tone, the ventricular pump function, the effective circulatory volume, or combinations thereof.

7. The system of claim 1, further comprising at least one sensor, wherein the at least one sensor can capture the external environment condition.

8. The system of claim 1, wherein the multivariate predictive model is configured to be trained by a machine learning algorithm.

9. A computer-implemented method, comprising:
classifying, using a multivariate predictive model, responses of a plurality of subjects to a stimulus, wherein the stimulus is selected from the group consisting of a physical stress, a disease-related stress, a severity of stress, a severity of insult, an external environment condition, and combinations thereof;
predicting at least one of a response or an intervention of a target subject to the stimulus based on the classified responses; and
adjusting a tissue metabolic adequacy of the target subject based on the predicted response or the predicted intervention of the target subject, wherein the tissue metabolic adequacy is calculated based on a vasomotor tone, a ventricular pump function, an effective circulatory volume, or combinations thereof.

10. The computer-implemented method of claim 9, wherein the stimulus comprises the external environment condition and wherein the external environment condition includes at least one of altitude, temperature, humidity, gravity, or combinations thereof.

11. The computer-implemented method of claim 9, wherein the response or the intervention of the target subject comprises a phenotype ranging from a robustness to a fragility of the subject.

12. The computer-implemented method of claim 9, further comprising calculating a tissue metabolic adequacy operating region based on at least one of the responses, the stimulus, the vasomotor tone, the ventricular pump function, an effective cardiac ejection, the effective circulatory volume, or combinations thereof.

13. The computer-implemented method of claim 9, further comprising maintaining a cardiovascular stability of the subject using an open-loop feedback control system, wherein the open-loop feedback control system is configured to measure and adjust at least one of the vasomotor tone, the ventricular pump function, the effective circulatory volume, or combinations thereof.

14. The computer-implemented method of claim 9, further comprising sensing the external environment condition using at least one sensor.

15. The computer-implemented method of claim 9, further comprising training the multivariate predictive model using a machine learning algorithm.

16. The computer-implemented method of claim 9, further comprising adjusting the tissue metabolic adequacy of the target subject by administering an effective amount of an active agent.
